(19) United States
(12) Patent Application Publication
(10) Pub. No.: US 2020/0172467 A1
(43) Pub. Date: Jun. 4, 2020

(54) EPOXY METHACRYLATE COMPOUNDS AND USE THEREOF

(71) Applicant: Hilti Aktiengesellschaft, Schaan (LI)

(72) Inventors: Georg Nickerl, Diessen am Ammersee (DE); Beate Gnass, Gersthofen (DE); Jens Bunzen, Augsburg (DE); Gerald Gaefke, Augsburg (DE); Thomas Bürgel, Landsberg (DE)

(73) Assignee: Hilti Aktiengesellschaft, Schaan (LI)

(21) Appl. No.: 16/613,182

(22) PCT Filed: Jun. 19, 2018
(86) PCT No.: PCT/EP2018/066215

§ 371 Date: Nov. 13, 2019

(30) Foreign Application Priority Data
Jul. 3, 2017 (EP) 17179290.6

(51) Int. Cl. C07C 69/54; C08L 33/10

(52) U.S. Cl. CPC C07C 69/54; C08L 33/10

(57) ABSTRACT

Low-viscosity epoxy methacrylate compounds are useful for lowering the viscosity of reactive resins and for reducing the forces for extruding a reactive-resin component containing these compounds. Furthermore, the low-viscosity epoxy methacrylate compounds are useful for chemical fastening.

The invention relates to low-viscosity epoxy methacrylate compounds as backbone resins, and to the use thereof in reactive resins, especially for lowering the viscosity of reactive resins containing such compounds and thus of the forces for extruding reactive-resin components produced therefrom. Furthermore, the invention relates to the use of these reactive resins and of their reactive-resin components for construction purposes, especially for chemical fastening.

The free-radical-curing fastening caulks currently in use are based on unsaturated polyesters, vinyl ester urethane resins and epoxy acrylates. These are mostly two-component reactive-resin systems, wherein one component is the resin (known as component (A)) and the other component (component (B)) contains the curing agent. Further ingredients such as inorganic fillers and additives, accelerators, stabilizers and reactive diluents may be contained in the one and/or the other component. By mixing the two components, the curing of the mixed components is initiated. During use of the fastening caulks for fastening of anchoring elements in drilled holes, the curing takes place in the drilled holes.

Such a fastening caulk is known, for example, from DE 3940138 A1. This describes fastening caulks on the basis of monomers that carry cycloaliphatic groups and may additionally contain unsaturated polyester or vinyl ester resins. Such mortar caulks have relatively high viscosities, however, whereby their use use is limited, especially for the chemical fastening technique.

Relatively broad temperature ranges, from −25° C. to +45° C., for example, can occur on construction sites, depending on time of year and/or geographic location. Therefore not only the high viscosity of the curable fastening caulks described in the introduction but also their resulting thixotropic behavior during application can lead to problems. Therefore the area of use of such fastening caulks is subject to great demands, especially for use in various temperature ranges.

On the one hand, a sufficiently low viscosity of the caulk that it can be extruded should be ensured in the low-temperature range, so that the flow resistance of the caulk is not too high. Thus it should be ensured that the caulks can be injected, for example into the drilled hole, using a hand dispenser, for example. In particular, during the use of static mixers, a low viscosity is of importance for flawless mixing of the two components.

On the other hand, the caulk should be sufficiently stable in the higher temperature range, so that continued running of the individual components after release of pressure on the dispenser is prevented and that the caulk does not leak out of the drilled hole during overhead installation.

A further problem caused by temperature fluctuations is that the free-radical chain polymerization does not take place uniformly. Thus the cured fastening caulk has fluctuating/irregular and frequently inadequate homogeneity, which is manifested in fluctuations of the load ratings and frequently also in generally low load ratings. For example, at temperatures below 20° C., premature setting of the fastening caulk may occur due to an increase of the viscosity. Thereby the conversion in the free-radical chain polymerization is substantially smaller, thus contributing to a reduction of the load ratings.

Since temperature fluctuations on the construction site cannot be avoided, a need continues to exist for two-component reactive-resin systems that ensure homogeneity both at high and at low temperatures as well as reproducibility of the load ratings associated therewith.

In order to address the foregoing problems, the proportion of reactive diluents in the fastening caulks available on the market is increased, ultimately leading to reduction of the resin proportion in the caulk. Not uncommonly, the proportion of reactive diluents amounts to at least 50% relative to the reactive resin.

However, the increase of the proportion of reactive diluents also leads to some disadvantages, which become evident above all during application of the fastening caulk for fastening of anchoring means in drilled holes.

A considerable disadvantage is that the reduction of the proportion of highly viscous resin, which is essential for the performance capability of the caulk, negatively influences the performance capability of the cured fastening caulk.

A further disadvantage is greater shrinkage of the fastening caulk after curing, which may additionally influence the performance capability of the cured fastening caulk negatively. This is attributed to the fact that the contact between the cured fastening caulk and the undercuts, formed in the wall of the drilled hole during creation of the drilled hole, which become apparent in particular during use of percussion drills, is significantly reduced. This usually also prevents application of fastening caulks based on free-radical-curing compounds in diamond-drilled holes.

A further disadvantage is that, depending on type of reactive diluent, the proportion of volatile organic compounds (VOC) in the caulks may increase. This may lead to evaporation from the fastening caulk and/or the canister and possibly to a drop in performance of the cured fastening caulk that results from this. In addition, some of these compounds may also be hazardous to health and/or are therefore subject to mandatory labeling.

In addition, the number of usable reactive diluents is small, since only few available reactive diluents are on the market at present. Other than the free-radical-curing functional groups, the available reactive diluents have no or only a very limited choice of other functional groups and therefore often have only little influence on the property of the cured fastening caulk. This leads to the situation that the fastening caulks are being developed mostly for specific applications, such as certain temperature ranges, for example, or for application in specific substrates. This calls for an immense development effort in order to be able to address new and broader applications with the fastening caulks.

Heretofore special products have been produced, the formulations of which are adapted to the special application temperatures. Products indeed exist that are intended for a broad temperature range while still having the same properties over the entire range. Precisely in the boundary ranges, i.e. at low and at high temperatures, impairments must be expected either in processability, in curing of the caulk or in the properties of the cured caulk. No fastening caulk is known that covers a very broad temperature range without having to tolerate losses in the boundary ranges.

A need therefore exists for fastening caulks having properties capable of being influenced not by the use of reactive diluents but instead by the resin ingredient.

One object of the present invention is to influence the properties of a reactive-resin master batch as well as of a reactive resin produced therefrom in a manner attributable solely to the structure of the backbone resin but not to the presence of additional compounds, such as reactive diluents or additives, for example. Mainly, the object of the present invention is to control the properties of a two-component or multi-component reactive-resin system by means of the backbone resin it contains. In particular, it is an object of the present invention to provide fastening caulks, such as two-component or multi-component reactive-resin systems, for example, the viscosity of which depends less on the temperature of application of the fastening caulk, which have a low viscosity, especially at low temperatures, such as below 20° C., for example, and thus make it possible to supply reactive-resin systems, which have smaller extrusion forces at application temperatures below 20° C., especially at application temperatures below 10° C., and thus are more user-friendly than the conventional fastening systems.

A further object of the invention is to provide a fastening caulk that has lower forces to extrude the reactive-resin component than do conventional caulks.

Yet another object of the present invention is to provide a fastening caulk that avoids constituents posing a serious health hazard in the reactive-resin component and that optionally is also exempt from labeling. In particular, it is an object to reduce the proportion of reactive diluents in reactive resins for chemical fastening, without having to sacrifice their function or functions and positive effects on the cured fastening caulk.

Yet another object of the present invention is to provide a fastening caulk that is distinguished by good processability, curing behavior and small shrinkage over a broad temperature range.

These objects are solved by a compound according to claim 1, by the use thereof according to claims 8 and 9, by a reactive resin, containing these compounds, according to claim 10, by a reactive-resin components according to claim 11 and by a reactive-resin system according to claim 13.

Surprisingly, it has been found that, due to the use of certain low-viscosity epoxy methacrylate compounds as backbone resin, a broad temperature range is achieved in which the viscosity of a reactive resin containing these compounds and of a reactive-resin component obtainable therefrom remains largely uninfluenced by the temperatures.

It was surprising that the inventive compounds have low viscosity despite their relatively high molecular weight.

Advantageously, the present invention permits, in comparison with the conventional systems, low extrusion forces at low application temperatures in a reactive-resin system. Due to the use of low-viscosity epoxy methacrylate compounds as backbone resin in reactive resins, it has therefore become possible to reduce the forces for extruding a reactive-resin system not only at 20° C. but also at lower temperatures, for example at temperatures below 10° C., preferably below 5° C., without requiring a high proportion of reactive diluent for the purpose.

Furthermore, it has been found that it is possible, due to the use of certain low-viscosity epoxy methacrylate compounds, to reduce the proportion of reactive diluents in reactive resins for chemical fastening, without having to sacrifice their function or functions and positive effects on the cured fastening caulk, since the proportion of backbone resin can be increased. Hereby it is possible on the one hand to increase the load ratings of a cured caulk.

The invention is based on the knowledge that it is possible to replace the higher-viscosity resins used heretofore in fastening caulks by smaller, low-viscosity backbone resins, in order to lower the proportion of reactive diluents without having to sacrifice their functionality.

For better understanding of the invention, the following explanations of the reactive-resin production method and of the terminology used herein are considered to be useful.

The reactive-resin production method, explained here by means of the example of a 1,4-butanediol diglycidyl ether and methacrylic acid, typically takes place as follows:

### 1. Production of Backbone-Resin/Reactive-Resin Master Batch

1,4-Butanediol diglycidyl ether and methacrylic acid are reacted in the presence of a catalyst and of an inhibitor (used to stabilize the backbone resin formed by the polymerization, and frequently also called stabilizer or process stabilizer). In this process, the backbone resin is obtained.

The reaction mixture obtained after the end of the reaction is known as reactive-resin master batch. This is not worked up further, i.e. the backbone resin is not isolated.

### 2. Production of Reactive Resin

After completion of the reaction to the backbone resin, an accelerator-inhibitor system, i.e. a combination of one or more additional inhibitors and one or more accelerators and optionally a reactive diluent, is added to the reactive-resin master batch.

Hereby the reactive resin is obtained.

The accelerator-inhibitor system is used to adjust the reactivity of the reactive resin, i.e. to adjust the point in time up to which the reactive resin has not yet cured completely after addition of an initiator and up to which point in time a plugging caulk mixed in with the reactive resin therefore remains processable after mixing with the initiator.

The inhibitor the accelerator-inhibitor system may be identical to the inhibitor for the production of the backbone resin, provided this is also suitable for adjusting the reactivity, or it may be a different inhibitor if it does not possess both functions. As an example, 4-hydroxy-2,2,6,6-tetramethyl-piperidinyl-1-oxyl (TEMPOL) may be used as stabilizer and as inhibitor for adjustment of the reactivity.

### 3. Production of Reactive-Resin Component

In order to use the reactive resin for construction purposes, especially for chemical fastening, one or more inorganic aggregates, such as additives and/or fillers, are added after production of the reactive resin.

Hereby the reactive-resin component is obtained.

Within the meaning of the invention, the terms used:
“backbone resin” means a usually solid or highly viscous free-radical-curing polymerizable resin, which cures by polymerization (e.g. after addition of an initiator in the presence of an accelerator) and as a rule exists without reactive diluent and without further purification and thus may contain impurities;
“reactive master batch” means the reaction product of the reaction for production of the backbone resin, i.e. a mixture of backbone resin, reactive diluent and optionally further ingredients of the reaction mixture;
“reactive resin” means a mixture of reactive-resin master batch, at least one accelerator and at least one inhibitor (also referred to as accelerator-inhibitor system), at least one reactive diluent and optionally further additives; the reactive resin is typically liquid or viscous and may be further processed to a reactive-resin component; herein, the reactive resin is also referred to as “resin mixture”;
“inhibitor” means a substance that suppresses an undesired free-radical polymerization during the synthesis or storage of a resin or of a resin-containing composition (these substances are also referred to in professional circles as “stabilizer”) or that causes a time delay of free-radical polymerization of a resin after addition of an initiator (usually in conjunction with an accelerator) (these substances are also referred to in professional circles as “inhibitor”—the respective meaning of the term is apparent from the context);
“accelerator” means a reagent that participates with the initiator in a reaction, so that larger quantities of free radicals are already generated by the initiator at lower temperatures, or that catalyzes the decomposition reaction of the initiator;
“reactive diluent” means liquid or low-viscosity monomers and backbone resins, which dilute other backbone resins or the reactive-resin master batch and thereby impart the necessary viscosity for application thereof, which contain functional groups capable of reaction with the backbone resin and during polymerization (curing) become largely an ingredient of the cured caulk (e.g. of the mortar); reactive diluents are also called co-polymerizable monomers;
“reactive-resin component” means a liquid or viscous mixture of reactive resin and fillers as well as optionally further components, e.g. additives; typically, the reactive-resin component is one of the two components of a two-component reactive-resin system for chemical fastening;
“initiator” means a substance that forms reaction-initiating free radicals (usually in combination with an accelerator);
“hardener component” means a composition that contains an initiator for polymerization of a backbone resin; the hardener component may be solid or liquid and besides the initiator may contain a solvent as well as fillers and/or additives; typically, the hardener component in addition to the reactive-resin component is the other of the two components of a two-component reactive-resin system for chemical fastening;
“mortar caulk/fastening caulk” means the composition that is obtained by mixing the reactive-resin component with the hardener component and that may be used directly as such for chemical fastening;
“reactive-resin system” generally means a system that comprises components stored separately from one another, so that curing of the backbone resin contained in one component takes place only after mixing of the components;
“two-component system” or “two-component reactive-resin system” means a reactive-resin system that comprises two components stored separately from one another; a reactive-resin component (A) and a hardener component (B), so that curing of the backbone resin contained in the reactive-resin component takes place only after mixing of the two components;
“multi-component system” or “multi-component reactive-resin system” means a reactive-resin system that comprises several components stored separately from one another, including a reactive-resin component (A) and a hardener component (B), so that curing of the backbone resin contained in the reactive-resin component takes place only after mixing of all components;
“construction purposes” means any application for creation and maintenance or repair of building parts and building structures, as a polymer concrete, as a plastic-based coating caulk or as a cold-curing road marking; in particular, the reinforcement of building parts and building structures; for example walls, ceilings or floors, the fastening of building parts, such as panels or blocks, for example of stone, glass or plastic, on building parts or building structures, for example by adhesive bonding (constructional adhesive bonding) and quite particularly chemical fastening of anchoring means, such as anchor rods, bolts or the like in recesses, such as drilled holes;
“chemical fastening” means fastening (by substance-to-substance and/or interlocking joining) of anchoring means, such as anchor rods, bolts, rebars, screws or the like in recesses, such as drilled holes, especially in holes drilled in various substrates; specially mineral substrates, such as those on the basis of concrete; cellular concrete; brickwork; lime sandstone, sandstone, natural rock; glass and the like, and metallic substrates; such as those of steel;
“aliphatic hydrocarbon group” means acyclic and cyclic saturated or unsaturated hydrocarbon groups that are not aromatic (PAC, 1995, 67, 1307; Glossary of class names of organic compounds and reactivity intermediates based on structure (IUPAC Recommendations 1995));
“meth)acryl . . . / . . . (meth)actyl . . . ” means that both the “methacryl . . . / . . . methaccryl . . .” and the “acryl . . . / . . . acryl . . . ” compounds are intended; preferably, “methacryl . . . / . . . methacryl . . . ” compounds are intended in the present invention;
“a”, “an”, “any”, as the indefinite article preceding a class of chemical compounds, e.g. preceding the word “epoxy methacrylate”, means that at least one, i.e. one or more compounds included under this class of chemical compounds, e.g. various epoxy methacrylates, may be intended. In a preferred embodiment, only one individual compound is intended with this indefinite article;
“at least one” means numerically “one or more”. In a preferred embodiment, “a”, “an”, “any” is meant numerically with this term;
“contain” and “comprise” mean that still further ingredients may be present in addition to those mentioned. These terms are intended to be inclusive and therefore also encompass “consist of”. “Consist of” is intended conclusively and means that no further ingredients may be present. In a preferred embodiment, the terms “contain” and “comprise” mean the term “consist of”;
“approximately” or “circa” preceding a numerical value mean a range of ±5% of this value, preferably ±2% of this value, more preferably ±1% of this value, particularly preferably ±0% of this value (i.e. exactly this value);

a range limited by numbers means that the two extreme values and any value within this range are disclosed individually.

All standards cited in this text (e.g. DIN standards) were used in the version that was current on the date of filing of this Application.

A first subject matter of the invention is a compound of general formula (I)

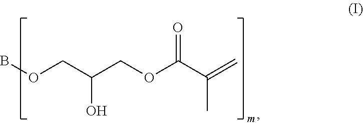

in which
   m is a whole number greater than or equal to 2, and
   B is a linear, branched or cyclic aliphatic hydrocarbon group.

A second subject matter is the use thereof for production of a reactive resin or a reactive-resin component for construction purposes. A third subject matter is the use thereof for lowering the viscosity of a reactive resin or of a reactive-resin component for construction purposes. A fourth subject matter is the use thereof for reducing the forces for extruding a reactive-resin component or a reactive-resin system. A fifth subject matter is a reactive resin comprising the compound of general formula (I), an inhibitor, an accelerator and optionally a reactive diluent. A sixth subject matter is a reactive-resin component for a reactive-resin system comprising the reactive resin. A seventh subject matter is a reactive-resin system, having the reactive-resin component and a hardener component, which contains an initiator. An eighth subject matter is the use of the reactive resin or of the reactive-resin system for construction purposes.

According to the invention, the low-viscosity epoxy methacrylate compound is a compound of general formula (I)

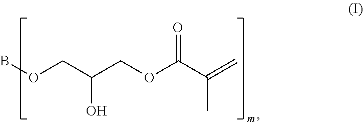

in which
   m is a whole number greater than or equal to 2, and
   B is a linear, branched or cyclic aliphatic hydrocarbon group.

The aliphatic hydrocarbon group B may be substituted, especially hydroxy-substituted. Preferably, the aliphatic hydrocarbon group is a linear or branched alkylene group, which optionally is hydroxy-substituted.

Preferably, the alkylene group is a C₂-C₁₂ alkylene group, more preferably a C₂-C₈ alkylene group and even more preferably a C₂-C₆ alkylene group. The alkylene group may be substituted, especially by an alkyl moiety.

Suitable alkylene groups are n-valent groups, as are obtained by removal of the glycidyl groups from an aliphatic polyglycidyl compound, wherein n stands for the number of glycidyl groups (valence) in the polyglycidyl compound. m corresponds to the said number of glycidyl groups in the polyglycidyl compound and thus to the valence of the polyglycidyl compound. The polyglycidyl compounds are derived from polyalcohols, in which the hydroxyl groups are converted entirely or partly by reaction with epihalohydrin to obtain a glycidyl ether group. Examples of suitable polyalcohols are 1,2-ethanedial, 1,2-propanediol, 1,3-propanediol, 1,3-butanediol, 1,4-butanediol, 1,6-hexanediol, glycerol, neopentyl glycol, ethylene glycol, cyclohexanedimethanol, trimethylolpropane, pentaerythritol and polyethylene glycols.

m is preferably a whole number between 2 and 5, more preferably between 2 and 4 and even more preferably 2 or 3.

If m is equal to 2, the compound of formula 1 has two terminal methacrylate groups, which may polymerize by free-radical reaction.

If m is greater than 2, the compound of formula 1 has more than two terminal methacrylate groups, which may polymerize by free-radical reaction. Due to the additional methacrylate group, for example a third methacrylate group when m is equal to 3, a branch point is obtained, and so a higher degree of branching can be achieved. Hereby the possibility of greater cross-linking is created. It is expected that a more greatly branched polymer network will be formed. This is advantageous in particular when trifuntional or multifunctional reactive diluents are to be avoided but the possibility of cross-linking is to be maintained.

Preferred inventive compounds with two methacrylate groups have the structure (II), (Ill), (IV) and (V):

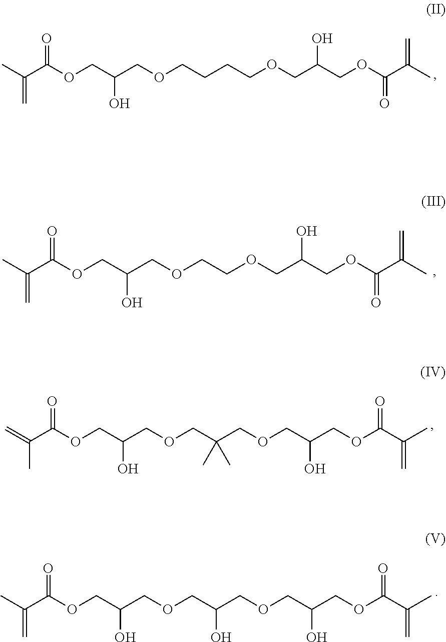

A preferred inventive compound with three methacrylate groups has the structure (VI):

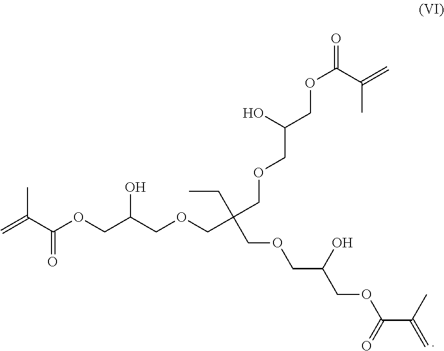 [SMILES: C=C(C)C(=O)OCC(O)COCC(CC)(COCC(O)COC(=O)C(=C)C)COCC(O)COC(=O)C(=C)C]

The inventive, low-viscosity epoxy methacrylate compounds may be obtained by reaction of methacrylic acid with a multifunctional glycidyl ether. Expediently, 0.7 to 1.2 carboxy equivalents of methacrylic acid are used per epoxy equivalent. The organic compounds containing epoxy groups and the methacrylic acid are then preferably used in approximately stoichiometric ratios, i.e. approximately one equivalent of methacrylic acid is used per epoxy equivalent of the organic compound. The glycidyl ether and the methacrylic acid are made to react in the presence of a catalyst and optionally of an inhibitor, which acts to stabilize the resulting backbone resin. In this process, the backbone resin is obtained.

Suitable glycidyl ethers are aliphatic or alicyclic glycidyl ethers from polyols (multihydric alcohols) having an epoxy functionality of at least 2, such as, for example, 1,4-butanediol diglycidyl ether (BDDGE), cyclohexanedimethanol diglycidyl ether, hexanediol diglycidyl ether and/or especially triglycidyl or higher glycidyl ethers, e.g. glycerol triglycidyl ether, pentaerythritol tetraglycidyl ether or trimethylolpropane triglycidyl ether (TMPTGE).

Beyond this, low-viscosity glycidyl ethers may be used that may be employed as reactive diluents in epoxy-amine systems and that have an epoxy functionality of at least two.

The inventive compounds of general formula (I) may be used, individually or as a mixture of at least two compounds, as the backbone resin in reactive-resin compositions, such as reactive resins and reactive-resin components. If several compounds of general formula (I) are used as backbone resin, the structure of the resulting polymer network may be influenced via the choice of compounds and the proportion of compounds in which m is greater than 2.

The said inventive compounds may be used alone or in addition to other resins commonly used for the respective purpose of application of the reactive resin. In this way the cross-linking density of the polymer network may be optionally influenced.

For the case that not all glycidyl groups are converted during production of the inventive compounds, or that some of the glycidyl groups are opened prior to the reaction, for example by a side reaction, compounds are obtained which may be present either as main compounds or as impurities in the reactive-resin master batch. To the extent that these compounds may be used for the inventive purposes, they are also comprised by the invention.

The compounds of general formula (I) are used according to the invention for production of a reactive resin. Hereby the viscosity of the reactive resin produced in this way may be lowered, without the need for a high proportion of reactive diluents, as is the case for commercial caulks, and without the problems associated with a high proportion of reactive diluents, such as, for example, reduction of the attainable load ratings of the cured caulk. Thus reduction of the forces for extruding a reactive-resin system containing the inventive compounds can be achieved.

The inventive reactive resin contains a compound of general formula (I) as described hereinabove as a backbone resin, an inhibitor, an accelerator and optionally a reactive diluent. Since the backbone resin, after its production, is typically used without isolation for production of the reactive resin, further ingredients, such as a catalyst, for example, contained in the reactive-resin master batch, are usually still also present in the reactive resin, besides the backbone resin.

The proportion of the compound of general formula (I) in the inventive reactive resin ranges from 25 wt % to 65 wt %, preferably from 30 wt % to 45 wt %, particularly preferably from 35 wt % to 40 wt % quite particularly preferably from 33 wt % to 40 wt % relative to the total weight of the reactive resin.

The stable free radicals that are commonly used for free-radical-curing polymerizable compounds, such as N-oxyl free radicals, as are known to the person skilled in the art, are suitable as inhibitors.

The inhibitor may function on the one hand to suppress undesired free-radical polymerization during synthesis of the backbone resin or during storage of the reactive resin and of the reactive-resin component. It may also function—optionally additionally—to cause a time delay of the free-radical polymerization of the backbone resin after addition of the initiator, and thereby to adjust the processing time of the reactive resin or of the reactive-resin component after mixing with the curing agent.

As examples of stable N-oxyl radicals, such may be used as described in DE 199 56 509 A1 and DE 195 31 649 A1. Such stable nitroxyl free radicals are of the piperidinyl-N-oxyl or tetrahydropyrrole-N-oxyl type or a mixture thereof.

Preferred stable nitroxoxyl free radicals are selected from the group consisting of 1-oxyl-2,2,6,6-tetramethylpiperidine, 1-oxyl-2,2,6,6-tetramethylpiperidine-4-ol (also known as TEMPOL), 1-oxyl-2,2,6,6-tetramethylpiperidine-4-one (also known as TEMPON), 1-oxyl-2,2,6,6-tetramethyl-4-carboxyl-piperidine (also known as 4-carboxy-TEMPO), 1-oxyl-2,2,5,5-tetramethylpyrrolidine, 1-oxyl-2,2,5,5-tetramethyl-3-carboxylpyrrolidine (also known as 3-carboxy-PROXYL) and mixtures of two or more of these compounds, wherein 1-oxyl-2,2,6,6-tetramethylpiperidine-4-ol (TEMPOL) is particularly preferred.

Besides the nitroxyl free radical of the piperidinyl-N-oxyl or tetrahydropyrrole-N-oxyl type, one or more further inhibitors may be present not only for further stabilization of the reactive resin or of the reactive-resin component (A) containing the reactive resin or of other compositions containing the reactive resin but also for adjustment of the resin reactivity.

The inhibitors that are commonly used for free-radical-curing polymerizable compounds, as are known to the person skilled in the art, are suitable for this purpose. Preferably, these further inhibitors are selected from among phenolic compounds and non-phenolic compounds and/or phenothiazines.

Phenols, such as 2-methoxyphenol, 4-methoxyphenol, 2,6-di-tert-butyl-4-methylphenol, 2,4-di-tert-butylphenol, 2,6-di-tert-butylphenol, 2,4,6-trimethylphenol, 2,4,6-tris(dimethylaminomethyl)phenol, 4,4′-thio-bis(3-methyl-6-tert-butylphenol), 4,4′-isopropylidenediphenol, 6,6′-di-tert-butyl-4,4′-bis(2,6-di-tert-butylphenol), 1,3,5-trimethyl-2,4,6-tris(3,5-di-tert-butyl-4-hydroxybenzyl)benzene, 2,2′-methylene-di-p-cresol, catechols, such as pyrocatechol, and catechol derivatives, such as butyl pyrocatechols, such as 4-tert-butyl pyrocatechol and 4,6-di-tert-butyl pyrocatechol, hydroquinones, such as hydroquinone, 2-methylhydroquinone, 2-tert-butylhydroquinone, 2,5-di-tert-butylhydroquinone, 2,6-di-tert-butylhydroquinone, 2,6-dimethylhydroquinone, 2,3,5-trimethylhydroquinone, benzoquinone, 2,3,5,6-tetrachloro-1,4-benzoquinone, methylbenzoquinone, 2,6-dimethylbenzoquinone, naphthoquinone, or mixtures of two or more thereof, are suitable as phenolic inhibitors. These inhibitors are often ingredients of commercial free-radical curing reactive-resin components.

Phenothiazines, such as phenothiazine and/or derivatives or combinations thereof, or stable organic free radicals, such as galvinoxyl and N-oxyl free radicals, for example, but not of piperidinyl-N-oxyl or tetrahydropyrrole-N-oxyl type, such as aluminum-N-nitrosophenylhydroxylamine, diethylhydroxylamine, oximes, such as acetaldoxime, acetone oxime, methyl ethyl ketoxime, salicyloxime, benzoxime, glyoximes, dimethylglyoxime, acetone-O-(benzyloxycarbonyl)oxime and the like, may be preferably regarded as non-phenolic inhibitors.

Furthermore, pyrimidinol or pyridinol compounds substituted in para position relative to the hydroxyl group may be used as inhibitors, as described in Patent Specification DE 10 2011 077 248 B1.

Preferably, the further inhibitors are selected from the group of catechols, catechol derivatives, phenothiazines, tert-butylcatechol, Tempol or a mixture of two or more thereof. Particularly preferably, the further inhibitors are selected from the group comprising catechols and phenothiazines. The further inhibitors used in the examples are quite particularly preferred, preferably approximately in the quantities specified in the examples.

Depending on the desired properties of the reactive resin, the further inhibitors may be used either alone or as a combination of two or more thereof.

The inhibitor or the inhibitor mixture is added in the proportions common in the art, preferably in a proportion of approximately 0.0005 to approximately 2 wt % (relative to the reactive resin ultimately produced therewith), more preferably of approximately 0.01 to approximately 1 wt % (relative to the reactive resin), even more preferably from approximately 0.05 to approximately 1 wt % (relative to the reactive resin), even much more preferably from approximately 0.2 to approximately 0.5 wt % (relative to the reactive resin).

The compounds of general formula (I), especially for use in reactive resins and reactive-resin components for chemical fastening and structural adhesive bonding, are generally cured by peroxides as curing agents. The peroxides are preferably initiated by an accelerator, so that polymerization takes place even at low application temperatures. The accelerator is already added to the reactive resin.

Suitable accelerators known to the person skilled in the art are, for example, amines, preferably tertiary amines and/or metal salts.

Suitable amines are selected from among the following compounds: dimethylamine, trimethylamine, ethylamine, diethylamine, triethylamine, n-propylamine, di-n-propylamine, tri-n-propylamine, isopropylamine, diisopropylamine, triisopropylamine, n-butylamine, isobutylamine, tert-butylamine, di-n-butylamine, diisobutylamine, tri-isobutylamine, pentylamine, isopentylamine, diisopentylamine, hexylamine, octylamine, dodecylamine, laurylamine, stearylamine, aminoethanol, diethanolamine, triethanolamine, aminohexanol, ethoxyaminoethane, dimethyl-(2-chloroethyl)amine, 2-ethylhexylamine, bis-(2-chloroethyl)amine, 2-ethylhexylamine, bis-(2-ethylhexyl)amine, N-methylstearylamine, dialkylamines, ethylenediamine, N,N′-dimethylethylenediamine, tetramethylethylenediamine, diethylenetriamine, permethyldiethylenetriamine, triethylenetetramine, tetraethylenepentamine, 1,2-diaminopropane, di-propylenetriamine, tripropylenetetramine, 1,4-diaminobutane, 1,6-diaminohexane, 4-amino-1-diethylaminopentane, 2,5-diamino-2,5-dimethylhexane, trimethylhexamethylenediamine, N,N-dimethylaminoethanol, 2-(2-diethylaminoethoxy)ethanol, bis-(2-hydroxyethyl)-oleylamine, tris-[2-(2-hydroxy-ethoxy)-ethyl]amine, 3-amino-1-propanol, methyl-(3-aminopropyl) ether, ethyl-(3-aminopropyl) ether, 1,4-butanediol-bis(3-aminopropyl ether), 3-dimethylamino-1-propanol, 1-amino-2-propanol, 1-diethylamino-2-propanol, diisopropanolamine, methyl-bis-(2-hydroxypropyl)amine, tris-(2-hydroxypropyl)amine, 4-amino-2-butanol, 2-amino-2-methyl propanol, 2-amino-2-methyl-propanediol, 2-amino-2-hydroxymethylpropanediol, 5-aiethylamino-2-pentanone, 3-methylamino-propionic acid nitrile, 6-aminohexanoic acid, 11-aminoundecanoic acid, 6-aminohexanoic acid ethyl ester, 11-aminohexanoic acid isopropyl ester, cyclohexylamine, N-methylcyclohexylamine, N,N-dimethylcyclohexylamine, dicyclohexylamine, N-ethylcyclohexylamine, N-(2-hydroxyethyl)-cyclohexylamine, N,N-bis-(2-hydroxyethyl)-cyclohexylamine, N-(3-aminopropyl)-cyclohexylamine, aminomethylcyclohexane, hexahydrotoluidine, hexahydrobenzylamine, aniline, N-methylaniline, N,N-dimethylaniline, N,N-diethylaniline, N,N-dipropylaniline, isobutylaniline, toluidine, diphenylamine, hydroxyethylaniline, bis-(hydroxyethyl)aniline, chloroaniline, aminophenols, aminobenzoic acids and their esters, benzylamine, dibenzylamine, tribenzylamine, methyldibenzylamine, a-phenylethylamine, xylidine, diisopropylaniline, dodecylaniline, aminonaphthalene, N-methylaminonaphthalene, N,N-dimethylaminonaphthalene, N, N-dibenzylnaphthalene, diaminocyclohexane, 4,4′-diamino-dicyclohexylmethane, diamino-dimethyl-dicyclohexylmethane, phenylenediamine, xylylenediamine, diaminobiphenyl, naphthalenediamines, toluidines, benzidines, 2,2-bis-(aminophenyl)-propane, aminoanisoles, amino-thiophenols, aminodiphenyl ether, aminocresols, morpholine, N-methylmorpholine, N-phenylmorpholine, hydroxyethylmorpholine, N-methylpyrrolidine, pyrrolidine, piperidine, hydroxyethylpiperidine, pyrroles, pyridines, quinolines, indoles, indolenines, carbazoles, pyrazoles, imidazoles, thiazoles, pyrimidines, quinoxalines, aminomorpholine, dimorpholinethane, [2,2,2]-diazabicyclooctane and N,N-dimethyl-p-toluidine.

According to the invention, di-iso-propanol-p-toluidine or N,N-bis(2-hydroxyethyl)-m-toluidine is used as accelerator.

Preferred amines are aniline derivatives and N,N-bisalkylarylamines, such as N,N-dimethylaniline, N,N-diethylaniline, N,N-dimethyl-p-toluidine, N,N-bis(hydroxyalkyl)arylamines, N,N-bis(2-hydroxyethyl)anilines, N,N-bis(2-hydroxyethyl)toluidine, N,N-bis(2-hydroxypropyl)aniline, N,N-bis(2-hydroxypropyl)toluidine, N,N-bis(3-methacryloyl-2-hydroxypropyl)-p-toluidine, N, N-dibutoxyhydroxypropyl-p-toluidi ne and 4,4′-bis(dimethylamino)diphenylmethane. Di-iso-propanol-p-toluidine is particularly preferred.

Polymeric amines, such as those obtained by polycondensation of N,N-bis(hydroxyalkyl)aniline with dicarboxylic acids or by polyaddition of ethylene oxide or other epoxides and these amines, are likewise suitable as accelerators.

Suitable metal salts are, for example, cobalt octoate or cobalt naphthenoate as well as vanadium, potassium, calcium, copper, manganese or zirconium carboxylates. Further suitable metal salts are the tin catalysts described hereinabove.

If an accelerator is used, it is introduced in a proportion of 0.01 to 10 wt %, preferably 0.2 to 5 wt % relative to the reactive resin.

The reactive resin may also contain a reactive diluent, if this is necessary.

Suitable reactive diluents are low-viscosity, free-radical-co-polymerizable compounds, preferably compounds exempt from labeling, which are added if necessary in order, among other purposes, to adapt the viscosity of the epoxy methacrylate or of the precursors during the production thereof.

Suitable reactive diluents are described in the Applications EP 1 935 860 A1 and DE 195 31 649 A1. Preferably, the reactive resin (the resin mixture) contains, as reactive diluent, a (meth)acrylic acid ester, wherein aliphatic or aromatic C₅-C₁₅ (meth)acrylates are selected particularly preferably. Suitable examples include: 2-hydroxypropyl (meth)acrylate, 3-hydroxypropyl (meth)acrylate, 1,2-ethanedial di-(meth)acrylate, 1,3-propanediol dimethacrylate, 1,3-butanediol di(meth)acrylate, 1,4-butanediol di(meth)acrylate, trimethylolpropane tri(meth)acrylate, phenylethyl (meth)acrylate, tetrahydrofurfuryl (meth)acrylate, ethyl triglycol (meth)acrylate, N,N-dimethylaminoethyl (meth)acrylate, N,N-dimethylaminomethyl (meth)acrylate, N,N-diethylaminoethyl (meth)acrylate, acetoacetoxyethyl (meth)acrylate, isobornyl (meth)acrylate, 2-ethylhexyl (meth)acrylate, tert-butylcyclohexyl (meth)acrylate, benzyl (meth)acrylate, methyl (meth)acrylate, n-butyl (meth)acrylate, iso-butyl (meth)acrylate, 3-trimethoxysilylpropyl (meth)acrylate, isodecyl (meth)acrylate, diethylene glycol di(meth)acrylate, triethylene glycol di(meth)acrylate, methoxypolyethylene glycol mono(meth)acrylate, trimethylcyclohexyl (meth)acrylate, 2-hydroxyethyl (meth)acrylate, dicyclopentenyloxyethyl (meth)acrylate and/or tricyclopentadienyl di(meth)acrylate, bisphenol A (meth)acrylate, novolac epoxy di(meth)acrylate, di-[(meth)acryloyl-maleoyl]-tricyclo-5.2.1.0.2.6-decane, 3-(meth)acryloyl-oxymethyl-tricylo-5.2.1.0.2.6-decane, 3-(meth)cyclo-pentadienyl (meth)acrylate and decalyl-2-(meth)acrylate; PEG di(meth)acrylate, such as PEG200 di(meth)acrylate, tetraethylene glycol di(meth)acrylate, solketal (meth)acrylate, cyclohexyl (meth)acrylate, phenoxyethyl di(meth)acrylate, 2-phenoxyethyl (meth)acrylate, hexanedio1,1,6-di(meth)acrylate, 1,2-butanediol di(meth)acrylate, methoxyethyl (meth)acrylate, butyldiglycol (meth)acrylate, tert-butyl (meth)acrylate and norbornyl (meth)acrylate. Methacrylates are preferred over acrylates.

2- and 3-Hydroxypropyl methacrylate, 1,2-ethanediol dimethacrylate, 1,4-butanediol dimethacrylate, 1,3-butanediol dimethacrylate, glycerol dimethacrylate, trimethylolpropane trimethacrylate, acetoacetoxyethyl methacrylate, isobornyl methacrylate, bisphenol A dimethacrylate, ethoxylated bisphenol A methacrylates such as E2BADMA or E3BADMA, trimethylcyclohexyl methacrylate, 2-hydroxyethyl methacrylate, PEG200 dimethacrylate and norbornyl methacrylate are particularly preferred and a mixture of 2- and 3-hydroxypropyl methacrylate and 1,4-butanediol dimethacrylate or a mixture of these three methacrylates is quite particularly preferred.

The most preferred is a mixture of 2- and 3-hydroxypropyl methacrylate. In principle, other common free-radical-polymerizable compounds may also be used as reactive diluents, alone or in a mixture with the (meth)acrylic acid esters, e.g. methacrylic acid, styrene, α-methylstyrene, alkylated styrenes, such as tert-butylstyrene, divinylbenzene and vinyl as well as allyl compounds, wherein the representatives thereof that are exempt from labeling are preferred. Examples of such vinyl or allyl compounds are hydroxybutyl vinyl ether, ethylene glycol divinyl ether, 1,4-butanediol divinyl ether, trimethylolpropane divinyl ether, trimethylolpropane trivinyl ether, mono-, di-, tri-, tetra- and polyalkylene glycol vinyl ethers, mono-, di-, tri-, tetra- and polyalkylene glycol allyl ethers, adipic acid divinyl ester, trimethylolpropane diallyl ether and trimethylolpropane triallyl ether.

The reactive diluent or diluents is or are added in a proportion up to 65 wt %, preferably up to 60 wt %, further preferably up to 55 wt %, particularly preferably in proportions below 50 wt %, relative to the reactive resin.

An exemplary reactive resin comprises a compound of general formula (I)

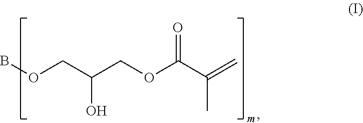

in which m is a whole number greater than or equal to 2, and B is a linear, branched or cyclic aliphatic hydrocarbon group, as the backbone resin, a stable nitroxyl radical as the inhibitor, a substituted toluidine as the accelerator and optionally a reactive diluent.

A preferred reactive resin comprises a compound of general formula (II)

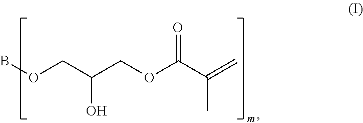

in which m is a whole number greater than or equal to 2, and B is a linear, branched or cyclic aliphatic hydrocarbon group, as the backbone resin, a stable nitroxyl radical as the inhibitor, a substituted toluidine as the accelerator and optionally a reactive diluent,

A further preferred reactive resin comprises a compound of formula (II), (Ill), (IV), (V) or (VI)

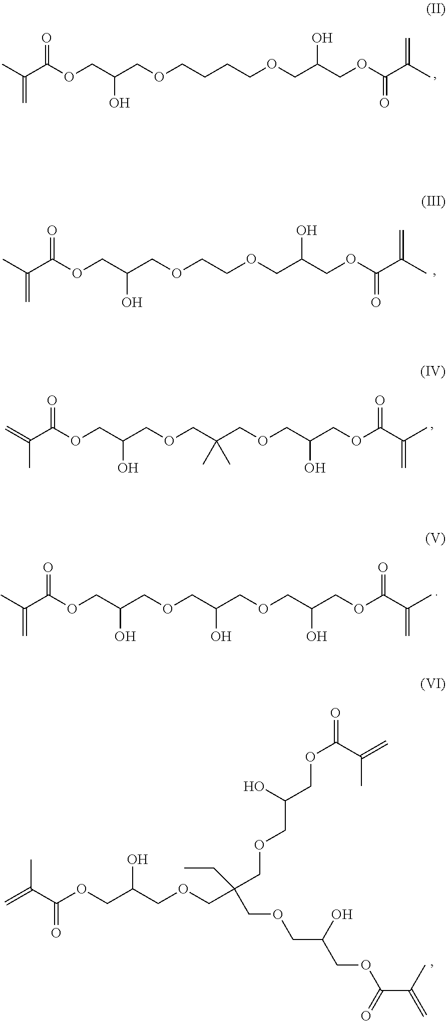

as the backbone resin, a stable nitroxyl radical as the inhibitor, a substituted toluidine as the accelerator and optionally a reactive diluent.

A particularly preferred reactive resin comprises a compound of the formula (II), (Ill), (IV), (V) or (VI) as the backbone resin, 4-hydroxy-2,2,6,6-tetramethyl-piperidinyl-1-oxyl (TEMPOL) as the inhibitor, di-iso-propanol-p-toluidine as the accelerator and a mixture of hydroxypropyl methacrylate (HPMA) and 1,4-butanediol dimethacrylate (BDDMA) as the reactive diluent.

By virtue of the low-viscosity backbone resin, an inventive reactive resin has a particularly low dynamic viscosity, and so it is possible to produce, for a reactive-resin system, a reactive-resin component, which exhibits substantially lower extrusion forces at application temperatures below 10° C., preferably at 0° C., than do conventional systems, without the high proportions of reactive diluents needed heretofore for the purpose.

A further subject matter of the invention is therefore a reactive-resin component that contains a reactive resin as just described. The reactive-resin component may contain inorganic aggregates, such as fillers and/or additives, in addition to the inventive reactive resin. It should be pointed out that some substances, both as fillers and optionally in modified form, may also be used as additive. For example, fumed silica functions more as a filler in its polar, non-post-treated form and more as an additive in its apolar, post-treated form. In cases in which exactly the same substance can be used as filler or additive, the total quantity thereof may not exceed the upper limit stipulated herein for fillers.

For production of a reactive-resin component for construction purposes, especially chemical fastening, common fillers and/or additives may be added to the inventive reactive resin. These fillers are typically inorganic fillers and additives, such as described hereinafter by way of example.

The proportion of the reactive resin in the reactive-resin component preferably ranges from approximately 10 to approximately 70 wt %, more preferably from approximately 30 to approximately 50 wt %, relative to the reactive-resin component. Accordingly, the proportion of fillers preferably ranges from approximately 90 to approximately 30 wt %, more preferably from approximately 70 to approximately 50 wt %, relative to the reactive-resin component.

Common fillers, preferably mineral or mineral-like fillers, such as quartz, glass, sand, quartz sand, quartz flour, porcelain, corundum, ceramic, talc, silica (e.g. fumed silica, especially polar non-post-treated fumed silica), silicates, aluminum oxides (e.g. alumina), clay, titanium dioxide, chalk, heavy spar, feldspar, basalt, aluminum hydroxide, granite or sandstone, polymeric fillers such as thermosetting plastics, hydraulically curable fillers, such as gypsum, burnt lime or cement (e.g. aluminate cement (often also referred to as aluminous cement) or Portland cement), metals, such as aluminum, carbon black, further wood, mineral or organic fibers or the like, or mixtures of two or more thereof, are used as fillers. The fillers may exist in any desired forms, for example as powder or flour or as shaped bodies, e.g. in the form of cylinders, rings, balls, platelets, rods, shells or crystals, or further in fiber form (fibrillar fillers), and the corresponding basic particles preferably have a maximum diameter of approximately 10 mm and a minimum diameter of approximately 1 nm. This means that the diameter is approximately 10 mm or any value smaller than approximately 10 mm, but larger than approximately 1 nm. Preferably the maximum diameter is a diameter of approximately 5 mm, more preferably of approximately 3 mm, even more preferably of approximately 0.7 mm. A maximum diameter of approximately 0.5 mm is quite particularly preferred. The more preferred minimum diameter is approximately 10 nm, even more preferably approximately 50 nm, quite particularly preferably approximately 100 nm. Diameter ranges obtained by combination of this maximum diameter and minimum diameter are particularly preferred. However, the globular inert substances (spherical shape), which have a distinctly more reinforcing effect, are preferred. Core-shell particles, preferably with spherical shape, may also be used as fillers.

Preferred fillers are selected from the group consisting of cement, silica, quartz, quartz sand. quartz flour and mixtures of two or more thereof. Fillers selected from the group consisting of cement, fumed silica, especially untreated, polar fumed silica, quartz sand, quartz flour and mixtures of two or more thereof are particularly preferred for the reactive-resin component (A). A mixture of cement (especially aluminate cement (often also referred to as aluminous cement) or Portland cement), fumed silica and quartz sand is quite particularly preferred for the reactive-resin component (A). For the hardener component (B), fumed silica is preferred as the sole filler or as one of several fillers; particularly preferably, not only fumed silica but also one or more further fillers are present.

Common additives, i.e. thixotropic agents, such as, optionally, organically or inorganically post-treated fumed silica (except if it is already being used as filler), especially apolarly post-treated fumed silica, bentonites, alkyl and methyl celluloses, castor oil derivatives or the like, plasticizers, such as phthalic acid or sebacic acid ester, further stabilizers in addition to the stabilizers and inhibitors used according to the invention, antistatic agents, thickening agents, flexibilizers, rheology additives, wetting agents, coloring additives, such as dyes or especially pigments, for example for different coloration of the components to permit better control of intermixing thereof, or the like, or mixtures of two or more thereof, are used as additives. Non-reactive diluents (solvents) may also be included, preferably in a proportion of up to 30 wt % relative to the total quantity of the reactive-resin component, such as lower alkyl ketones, e.g. acetone, di-lower-alkyl lower alkanoylamides, such as dimethylacetamide, lower alkylbenzenes, such as xylenes or toluene, phthalic acid esters or paraffins, water or glycols. Furthermore, metal scavengers in the form of surface-modified fumed silicas may be contained in the reactive-resin component. Preferably, at least one thixotropic agent is present as additive, particularly preferably an organically or inorganically post-treated fumed silica, quite particularly preferably an apolarly post-treated fumed silica.

In this respect, reference is made to the Applications WO 02/079341 and WO 02/079293 as well as WO 2011/128061 A1.

The proportion of additives in the reactive-resin component may range up to approximately 5 wt %, relative to the reactive-resin component.

The reactive resins produced according to the invention can be used in many areas, in which unsaturated polyester resins, vinyl ester resins or vinyl ester urethane resins are otherwise commonly used. They are commonly used as resin ingredient in the reactive-resin component of a reactive-resin system, such as a multi-component system, typically a two-component system comprising a reactive-resin component (A) and a hardener component (B). This multi-component system can exist in the form of a cartridge system, a canister system or a film-bag system. During use of the system as intended, the components are extruded from the cartridges, canisters or film bags either by application or mechanical forces or by gas pressure, mixed with one another, preferably using a static mixer, through which the ingredients are conveyed, and applied.

Subject matter of the present invention is therefore also a reactive-resin system having a reactive-resin component (A) and a hardener component (B) as just described, that contains an initiator for the epoxy methacrylate compound.

The initiator is customarily a peroxide. All peroxides known to the person skilled in the art that are used for curing of unsaturated polyester resins and vinyl ester resins may be employed. Such peroxides comprise organic and inorganic peroxides that are either liquid or solid, wherein hydrogen peroxide may also be used. Examples of suitable peroxides are peroxycarbonates (of the formula —OC(O)O—), peroxy esters (of the formula —C(O)OO—), diacyl peroxides (of the formula —O(O)OOC(O)—), dialkyl peroxides (of the formula —OO—) and the like. These may be present as oligomers or polymers.

Preferably, the peroxides are selected from the group of organic peroxides. Suitable organic peroxides are: tertiary alkyl hydroperoxides, such as tert-butyl hydroperoxide, and other hydroperoxides, such as cumene hydroperoxide, peroxy esters or peracids, such as tert-butyl peresters, benzoyl peroxide, peracetates and perbenzoates, lauryl peroxide, including (di)peroxy esters, perethers, such as peroxy diethyl ether, perketones, such as methyl ethyl ketone peroxide. The organic peroxides used as hardeners are often tertiary peresters or tertiary hydroperoxides, i.e. peroxide compounds with tertiary carbon atoms, which are bound directly to an —O—O-acyl- or —OOH— group. However, mixtures of these peroxides with other peroxides may also be used according to the invention. The peroxides may also be mixed peroxides, i.e. peroxides that have two different peroxide-carrying units in one molecule. Preferably, (di-benzoyl) peroxide (BPO) is used for curing.

The reactive-resin system may be present in the form of a two-component or multi-component system, in which the respective components exist spatially separated from one another, so that a reaction (curing) of the components take place only after they have been mixed.

A two-component reactive-resin system preferably comprises the A component and the B component separated, to ensure inhibition of reaction, into different containers, for example of a multi-chamber apparatus, such as a multi-chamber cartridge and/or canister, from which containers the two components are extruded by application of mechanical pressing forces or by application of a gas pressure and then mixed. A further possibility consists in packaging the two-component reactive-resin system as two-component capsules, which are introduced into the drilled hole and destroyed by percussively turning the fastening element to set it while simultaneously intermixing the two components of the mortar caulk. Preferably, a cartridge system or an injection system is used herein, in which the two components are extruded from the separated containers and passed through a static mixer, in which they are mixed homogeneously and then discharged via a nozzle, preferably directly into the drilled hole.

In a preferred embodiment of the inventive reactive-resin system, the reactive-resin system is a two-component system, and the reactive-resin component (A) contains not only the backbone resin but additionally also a hydraulically binding or polycondensable inorganic compound, especially cement, and the hardener component (B) contains not only the initiator for polymerization of the backbone resin but also water. Such hybrid mortar systems are described in detail in DE 4231161 A1. Therein, component (A) preferably contains cement as the hydraulically binding or polycondensable inorganic compound_{;} for example Portland cement or aluminous cement, wherein cements free of transition metal oxides or low in transition metals are particularly preferred. Gypsum as such or mixed with the cement may also be used as the hydraulically binding inorganic compound. Component (A) may also comprise, as the polycondensable inorganic compound, silicatic polycondensable compounds, especially substances containing soluble, dissolved and/or amorphous silicon dioxide, such as, for example, polar, non-post-treated fumed silica.

The volume ratio of component A to component B in a two-component system is preferably 3:1, 5:1 or 7:1. A volume ratio of 3:1 or 5:1 is particularly preferred.

In a preferred embodiment, the reactive-resin component (A) therefore contains the following:
at least one epoxy methacrylate as defined hereinabove, preferably a compound of formula (II), (III), (IV), (V) or (VI);
at least one inhibitor of piperidinyl-N-oxyl or tetrahydropyrrole-N-oxyl type as defined hereinabove, preferably TEMPOL;
at least one accelerator defined as hereinabove, preferably a toluidine derivative, particularly preferably di-iso-propanol-p-toluidine;
at least one hydraulically binding or polycondensable inorganic compound, preferably cement; and
at least one thixotropic agent, preferably fumed silica,

and the hardener component (B) contains:
at least one initiator for initiation of polymerization of the epoxy methacrylate, preferably benzoyl peroxide (BPO) or tert-butyl peroxybenzoate; and
water.

In a more preferred embodiment, the reactive-resin component (A) contains:
at least one epoxy methacrylate as defined hereinabove, preferably a compound of formula (II), (Ill), (IV), (V) or (VI);
at least one inhibitor of piperidinyl-N-oxyl or tetrahydropyrrole-N-oxyl type as defined hereinabove, preferably TEMPOL;
at least one accelerator, preferably a toluidine derivative, particularly preferably di-iso-propanol-p-toluidine;
at least one hydraulically binding or polycondensable inorganic compound, preferably cement; and
at least one thixotropic agent, preferably fumed silica,

and the hardener component (B) contains:
at least one initiator for initiation of polymerization of the epoxy methacrylate, preferably benzoyl peroxide (BPO) or tert-butyl peroxybenzoate;
at least one filler, preferably quartz sand or quartz flour; and
water.

In an even more preferred embodiment, the reactive-resin component (A) contains:
at least one epoxy methacrylate as defined hereinabove, preferably a compound of formula (II), (III), (IV), (V) or (VI);
at least one inhibitor of piperidinyl-N-oxyl or tetrahydropyrrole-N-oxyl type as defined hereinabove, preferably TEMPOL;
at least one accelerator, preferably a toluidine derivative, particularly preferably di-iso-propanol-p-toluidine;
at least one further inhibitor, which is selected from the group consisting of catechols and phenothiazines;
at least one hydraulically binding or polycondensable inorganic compound, preferably cement; and
at least one thixotropic agent, preferably fumed silica,

and the hardener component (B) contains:
at least one initiator for initiation of polymerization of the epoxy methacrylate, preferably benzoyl peroxide (BPO) or tert-butyl peroxybenzoate;
at least one filler, preferably quartz sand or quartz flour;
at least one thixotropic agent, preferably fumed silica; and
water.

In an even more preferred embodiment, the reactive-resin component (A) contains:
at least one epoxy methacrylate as defined hereinabove, preferably a compound of formula (II), (III), (IV), (V) or (VI);
at least one inhibitor of piperidinyl-N-oxyl or tetrahydropyrrole-N-oxyl type as defined hereinabove, preferably TEMPOL;
at least one accelerator, preferably a toluidine derivative, particularly preferably di-iso-propanol-p-toluidine;
at least one further inhibitor, which is selected from the group consisting of catechols and phenothiazines;
at least one hydraulically binding or polycondensable inorganic compound, preferably cement;
at least one thixotropic agent, preferably fumed silica; and
at least one further filler, preferably quartz sand,

and the hardener component (B) contains:
benzoyl peroxide (BPO) or tert-butyl peroxybenzoate as initiator for initiation of polymerization of the epoxy methacrylate;
at least one filler, preferably quartz sand or quartz flour;
at least one thixotropic agent, preferably fumed silica; and
water.

In an even more preferred embodiment, the reactive-resin component (A) contains:
at least one epoxy methacrylate as defined hereinabove, preferably a compound of formula (II), (III), (IV), (V) or (VI);
TEMPOL,
di-iso-propanol-p-toluidine;
at least one further inhibitor, which is selected from the group consisting of catechols and phenothiazines;
cement;
fumed silica; and
quartz sand,

and the hardener component (B) contains:
at least one initiator for initiation of polymerization of the epoxy methacrylate;
fumed silica;
quartz sand or quartz flour and
water.

In each of these embodiments, the reactive-resin component (A) additionally also contains, in a preferred embodiment, at least one reactive diluent. Preferably, this reactive diluent is a monomer or a mixture of several monomers of the backbone resin.

In each of these embodiments, the reactive-resin components (A) and the hardener components (B) can be combined with one another in any desired manner.

Such a reactive-resin system is used above all in the building sector (construction purposes), for example for creation and maintenance or repair of building parts and building structures, for example of concrete, as a polymer concrete, as a plastic-based coating caulk or as a cold-curing road marking, for reinforcement of building parts and building structures, for example walls, ceilings or floors, the fastening of building parts, such as panels or blocks, for example of stone, glass or plastic, on building parts or building structures, for example by adhesive bonding (constructional adhesive bonding). It is particularly suitable for chemical fastening. It is quite particularly suitable for chemical fastening (by substance-to-substance and/or interlocking joining) of anchoring means, such as anchor rods, bolts, rebars, screws or the like in recesses, such as drilled holes, especially in holes drilled in various substrates, especially mineral substrates, such as those on the basis of concrete, cellular concrete, brickwork, lime sandstone, sandstone, natural rock, glass and the like, and metallic substrates, such as those of steel. In one embodiment, the substrate of the drilled hole is concrete and the anchoring means consists of steel or iron. In a further embodiment, the substrate of the drilled hole is steel and the anchoring means consists of steel or iron. For this purpose, the components are injected into the drilled hole, after which the devices to be fastened, such as threaded anchor rods and the like, are introduced into the drilled hole charged with the curing reactive resin and are appropriately adjusted.

The invention will be further explained on the basis of the following examples.

### EXAMPLES

Reactive-resin master batches, reactive resins, reactive-resin components and two-component reactive-resin systems were produced as backbone resin using compounds (II) and (VI). The dynamic viscosity of the reactive resins and of the reactive-resin components were determined, as were the forces for extruding the two-component reactive-resin systems.

### A1. Production of Reactive-Resin Master Batch A1 with Compound (II)

645 g 1,4-Butanediol diglycidyl ether (Araldite® DY 026 SP; Huntsmann Advanced Materials), 518 g methacrylic acid (BASF SE), 6.0 g tetraethylammonium bromide (Merck KGaA Germany), 0.23 g phenothiazine (D Prills; Allessa Chemie) and 0.25 g 4-hydroxy-2,2,6,6-tetramethyl-piperidinyl-l-oxyl (TEMPOL; Evonik Industries AG) were introduced into a 2-liter glass laboratory reactor with internal thermometer and stirrer shaft. The batch was heated for 240 minutes at 100° C.

Hereby reactive-resin master batch A1 and containing the compound (II) as backbone resin was obtained. Compound (II) has the following structure:

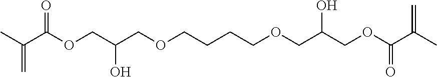 [SMILES: C=C(C)C(=O)OCC(O)COCCCCOCC(O)COC(=O)C(=C)C]

### A2. Production of Reactive Resin A2

6.5 g 4-Hydroxy-2,2,6,6-tetramethyl-piperidinyl-1-oxyl (TEMPOL; Evonik Degussa GmbH) and 26.25 g di-iso-propanol-p-toluidine (BASF SE) were added to a mixture of 489 g reactive-resin master batch A1, 489 g hydroxypropyl methacrylate and 489 g 1,4-butanediol dimethacrylate (BDDMA; Evonik AG).

Hereby reactive resin A2 containing compound (II) as backbone resin was obtained.

### A3. Production of Reactive-Resin Component A3

354 g Reactive resin A2 was mixed with 185 g Secar® 80 (Kerneos Inc.), 27 g CAB-O-SIL® TS-720 (Cabot Corporation) and 335 g quartz sand F32 (Quarzwerke GmbH) in the dissolver under vacuum, using a PC Labor System Dissolver of LDV 0.3-1 type. The caulk was stirred decentrally for 8 minutes at 3500 rpm under vacuum (p≤5. 100 mbar) with a 55 mm dissolver disk and an edge scraper.

Hereby reactive-resin component A3 was obtained.

### B1. Production of Reactive-Resin Master Batch B1 with Compound (VI)

840 g Trimethylolpropane triglycidyl ether, 742 g methacrylic acid, 17.5 g tetraethylammonium bromide, 0.33 g phenothiazine (D Prills; Allessa Chemie) and 0.36 g 4-hydroxy-2,2,6,6-tetramethyl-piperidinyl-1-oxyl (TEMPOL; Evonik Degussa GmbH) were introduced into a 2-liter glass laboratory reactor with internal thermometer and stirrer shaft. The batch was heated for 300 minutes at 100° C. Then 400 g 1,4-butanediol dimethacrylate (BDDMA; Evonik AG) was added.

Hereby reactive-resin master batch B1 containing compound (VI) as backbone resin was obtained. Compound (VI) has the following structure:

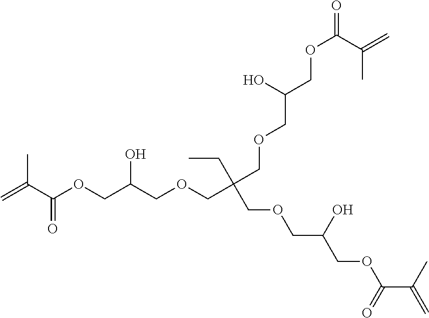 [SMILES: C=C(C)C(=O)OCC(O)COCC(CC)(COCC(O)COC(=O)C(=C)C)COCC(O)COC(=O)C(=C)C]

### B2. Production of Reactive Resin B2

6.0 g 4-Hydroxy-2,2,6,6-tetramethyl-piperidinyl-1-oxyl (TEMPOL; Evonik Degussa GmbH) and 22.8 g di-iso-propanol-p-toluidine (BASF SE) were added to a mixture of 530 g reactive-resin master batch B1, 424 g hydroxypropyl methacrylate and 318 g 1,4-butanediol dimethacrylate (BDDMA; Evonik AG).

Hereby reactive-resin B2 containing compound (VI) as backbone resin was obtained.

### B3. Production of Reactive-Resin Component B3

354 g Reactive resin B2 was mixed with 185 g Secar® 80 (Kerneos Inc.), 27 g CAB-O-SIL® TS-720 (Cabot Corporation) and 335 g quartz sand F32 (Quarzwerke GmbH) in the dissolver under vacuum, using a PC Labor System Dissolver of LDV 0.3-1 type, as indicated under heading A3.

Hereby reactive-resin component B3 was obtained.

### C1. Production of Comparison Reactive-Resin Master Batch with Comparison Compound 1

Comparison reactive-resin master batch C1 containing comparison compound 1 as backbone resin was synthesized according to the method in EP 0 713 015 A1, which is included herewith as reference and to the entire disclosure of which reference is made.

Hereby comparison reactive-resin master batch C1 containing 65 wt % comparison compound 1 as backbone resin and 35 wt % hydroxypropyl methacrylate, relative to the total weight of the comparison reactive-resin master batch, was obtained.

The product (comparison compound 1) has an oligomer distribution, wherein the oligomer containing a repeat unit has the following structure:

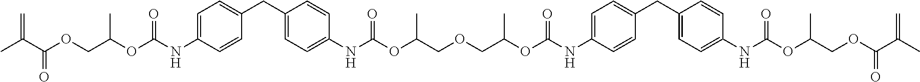

### C2. Production of Comparison Reactive Resin C2

9.2 g 4-Hydroxy-2,2,6,6-tetramethyl-piperidinyl-1-oxyl (TEMPOL; Evonik Industries AG) and 35.0 g di-iso-propanol-p-toluidine (BASF SE) were added to a mixture of 1004 g comparison reactive-resin master batch C1, 300 g hydroxypropyl methacrylate and 652 g 1,4-butanediol dimethacrylate (BDDMA; Evonik AG).

Hereby comparison reactive-resin C2 containing the comparison compound 1 as backbone resin was obtained.

### C3. Production of Comparison Reactive-Resin Component C3

354 g Comparison reactive resin C2 was mixed with 185 g Secar® 80 (Kerneos Inc.), 27 g CAB-O-SIL® TS-720 (Cabot Corporation) and 335 g quartz sand F32 (Quarzwerke GmbH) in the dissolver under vacuum, using a PC Labor System Dissolver of LDV 0.3-1 type, as indicated under heading A3.

Hereby comparison reactive-resin component C3 was obtained.

In order to demonstrate the influence of compounds (II) and (VI) on the viscosity of a reactive-resin master batch containing these compounds, of a reactive resin and of a reactive-resin component, the viscosity of the inventive reactive-resin component as well as the forces for extruding two-component reactive-resin systems were measured and respectively compared with the comparison reactive-resin component and the comparison two-component reactive-resin system.

### Measurement of the Dynamic Viscosity of the Reactive Resins

The dynamic viscosity of reactive resins A2 and B2 and of comparison reactive resin C2 was measured with a cone-and-plate measuring system according to DIN 53019. The diameter of the cone was 60 mm and the opening angle was 1°. The measurement was performed at a constant shear velocity of 150/s and a temperature of 23° C. (unless otherwise specified for the measured data). The measurement duration was 180 s and one measured point was generated every second. The shear velocity was attained by a preceding ramp from 0 to 150/s over a duration of 120 s. Since Newtonian fluids are involved, a linear evaluation over the measurement portion was undertaken and the viscosity was determined with constant shear velocity of 150/s over the measurement portion. Respectively three measurements were made, wherein the values indicated in Table 1 are the mean values of the three measurements.

### Measurement of the Dynamic Viscosity of the Reactive-Resin Components

The dynamic viscosity of reactive-resin components A3 and B3 and of comparison reactive-resin component C3 was measured with a cone-and-plate measuring system according to DIN 53019. The diameter of the plate was 20 mm and the gap distance was 3 mm. In order to prevent escape of the sample from the gap, a limiting ring of Teflon having a distance of 1 mm from the upper plate was used. The measurement temperature was 25° C. The method consisted of three portions: 1st Low shear, 2nd High shear, 3rd Low shear. During the 1st portion, shear was applied for 3 minutes at 0.5/s. In the 2nd portion, the shear velocity was increased logarithmically from 0.8/s to 100/s in 8 stages of 15 seconds each. These individual stages were: 0.8/s; 1.724/s; 3.713/s; 8/s; 17.24/s; 37.13/s; 80/s; 100/s. The 3rd portion was a repetition of the 1st portion. The viscosities were read at the end of each portion. The values summarized in Table 2 correspond to the value of the second portion at 100/s. Respectively three measurements were made, wherein the values indicated in Table 2 are the mean values of the three measurements.

### Measurement of the Forces for Extruding the Two-Component Reactive-Resin Systems

For determination of the extrusion forces at 0° C. and 25° C., the reactive-resin components (component (A)) and the hardener component (component (B)), produced as in the foregoing, of the commercially available product HIT-HY 110 (Hilti Aktiengesellschaft; batch number: 1610264) were filled into plastic canisters (Ritter GmbH; volume ratio A:B=3:1) with inside diameters of approximately 47 mm (component (A)) and respectively approximately 28 mm (component (B)) and adjusted to temperatures of 0° C. and 25° C. respectively. Using a material-testing machine of the Zwick Co. with a load cell (test range up to 10 kN), the canisters were extruded via a static mixer (HIT-RE-M mixer; Hilti Aktiengesellschaft) by with a constant speed of 100 mm/min over a path of 45 mm and in the process the mean force developed was measured.

The dynamic viscosity of reactive resins A2 and B2 was compared with the dynamic viscosity of comparison reactive resin C2. The results are compiled in Table 1.

TABLE 1
Results of measurement of the dynamic viscosity of reactive
resins A2 and B2 and of comparison reactive resin C2

| | | | Comparison |
|---|---|---|---|
| | Reactive | Reactive | reactive |
| | resin A2 | resin B2 | resin C2 |
| Viscosity | 22 | 28 | 70 |
| [mPa · s] | | | |

From the values in Table 1, it is evident that reactive resins A2 and B2, which contain the inventive compounds (II) and (IV) as backbone resin, have a much lower dynamic viscosity compared with the dynamic viscosity of the comparison resin C2, which contains comparison compound 1 as backbone resin.

The dynamic viscosity of reactive-resin components A3 and B3 was compared with the dynamic viscosity of comparison reactive-resin component C3. The measured values are summarized in Table 2.

TABLE 2
Results of the measurement of the dynamic viscosity
of reactive-resin components A3 and B3 and of
comparison reactive-resin component C3

| | | | Comparison |
|---|---|---|---|
| | Reactive resin | Reactive resin | reactive resin |
| | component A3 | component B3 | component C3 |
| Viscosity | 11.3 | 12.2 | 13.9 |
| [mPa · s] | | | |

The values in Table 2 show that reactive-resin components A3 and B3 produced from reactive resins A2 and B2 also have a low dynamic viscosity compared with the dynamic viscosity of comparison component C3 from comparison reactive resin C2.

The forces for extruding two-component reactive-resin systems containing the inventive reactive-resin components A3 and B4 were compared with the force for extruding the comparison two-component reactive-resin system, which contains comparison reactive-resin component C3. The values measured at 0° C. and at 25° C. are summarized in Table 3.

TABLE 3
Forces at 0° C. and at 25° C. for extruding two-component
reactive-resin systems containing reactive-resin components
A3 and B3 and the comparison two-component reactive-resin
system, which contains comparison reactive-resin component C3

| | | | Comparison |
|---|---|---|---|
| | | | reactive resin |
| | Reactive resin | Reactive resin | system with |
| | system with | system with | comparison |
| | reactive-resin | reactive-resin | reactive-resin |
| | component A3 | component B3 | component C3 |
| Force at 0° C. [N] | 1203 | 1270 | 1631 |
| Force at 25° C. [N] | 843 | 959 | 1151 |

The results in Table 3 show that the two-component reactive-resin systems, which contain the inventive compounds (II) and (VI) as backbone resins, exhibit much lower extrusion forces at 25° C. and also at 0° C. than does the comparison two-component reactive-resin system, which contains comparison compound 1 as backbone resin.

1. A compound of the general formula (1) 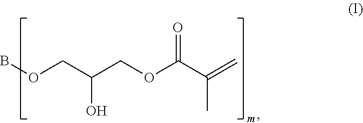
in which
m is a whole number greater than or equal to 2, and
B is a linear, branched or cyclic aliphatic hydrocarbon group.

2. The compound according to claim 1, wherein the aliphatic hydrocarbon group B is substituted.

3. The compound according to claim 2, wherein the aliphatic hydrocarbon group B is hydroxy-substituted.

4. The compound according to claim 1, wherein the aliphatic hydrocarbon group is a linear or branched C₂-C₁₂ alkylene group.

5. The compound according to claim 4, wherein the aliphatic hydrocarbon group is a linear or branched C₂-C₈ alkylene group.

6. The compound according to claim 1, wherein m is 2 or 3 and the aliphatic hydrocarbon group is a linear or branched C₂-C₈ alkylene group.

7. A method of construction, comprising:
incorporating the compound according to claim 1 in a reactive resin or a reactive-resin component.

8. A method of lowering a viscosity in a reactive-resin component for construction purposes, the method comprising:
incorporating the compound of claim 1 in a reactive-resin component for construction purposes in need thereof.

9. A method of reducing a force for extruding a reactive-resin component or a reactive-resin system, the method comprising:
incorporating the compound according to claim 1 in a reactive-resin component or a reactive-resin system in need thereof.

10. A reactive resin, comprising:
the compound according to claim 1,
an inhibitor,
an accelerator, and
optionally a reactive diluent.

11. A reactive-resin component, comprising:
the reactive resin according to claim 10.

12. A reactive-resin system, comprising:
the reactive-resin component according to claim 11 and
a hardener component.

13. The reactive-resin system according to claim 12, wherein the reactive-resin component and/or the hardener component contains at least one inorganic filler and/or an inorganic additive.

14. (canceled)

15. (canceled)

16. A method of construction with the reactive resin according to claim 10, the method comprising:
combining at least the compound, the inhibitor, and the accelerator, thereby obtaining the reactive resin, and
applying the reactive resin for construction.

17. A method of construction with the reactive-resin system according to claim 12, the method comprising:
combining the reactive-resin component and the hardener component, thereby obtaining the reactive-resin system, and
applying the reactive-resin system for construction.

18. A method for chemically fastening an anchor in a drilled hole with the reactive resin according to claim 10, the method comprising:
combining at least the compound, the inhibitor, and the accelerator, thereby obtaining the reactive resin, and
chemically fastening the anchor in the drilled hole with the reactive resin.

19. A method for chemically fastening an anchor in a drilled hole with the reactive-resin system according to claim 12, the method comprising:
combining the reactive-resin component and the hardener component, thereby obtaining the reactive-resin system, and
chemically fastening the anchor in the drilled hole with the reactive-resin system.
